(19) United States
(12) Patent Application Publication
(10) Pub. No.: US 2010/0068166 A1
(43) Pub. Date: Mar. 18, 2010

(54) ORAL COMPOSITION COMPRISING DIMETHICONE COPOLYOL

(75) Inventor: Christabel Fowler, Weybridge, Surrey (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: GLAXO GROUP LIMITED, Greenford, Middlesex (GB)

(21) Appl. No.: 11/917,975

(22) PCT Filed: Jun. 28, 2006
(86) PCT No.: PCT/EP2006/006408

§ 371 Date: Dec. 18, 2007

(30) Foreign Application Priority Data
Jun. 30, 2005 (GB) 0513367.3

(51) Int. Cl. A61K 31/80; A61P 43/00

(52) U.S. Cl. 424/78.02

(57) ABSTRACT

Oral compositions comprising silicone polyols are described for lubricating and hydrating the buccal cavity and are of benefit in alleviating the symptoms of xerostomia (dry mouth).

The present invention relates to oral compositions comprising silicone polyols for lubricating the buccal cavity.

Lubrication of the buccal cavity leads to an improved mouth feel and can help to combat problems associated with dry mouth.

Lack of lubrication of the buccal cavity can lead to difficulties in speaking, swallowing and masticating, and sleeping, and the problems of polydypsia and polyuria, (Kelly., et al, Bioadhesive, rheological, lubricant and other aspects of an oral gel formulation intended for the treatment of xerostomia. International Journal of Pharmaceutics, 2004; 278: 391-406).

Dry mouth can result from several causes including dehydration, a reduction in fluid intake, medications, systemic disease and radiotherapy of the head and neck, (Frost., et al, Patient preferences in a preliminary study of comparing an intra-oral lubricating device with the usual dry mouth lubricating methods. British Dental Journal, 2002; 193: 403-408). In particular, xerostomia or dry mouth, is a condition in which an excessive dryness within the buccal cavity occurs.

Xerostomia is often a contributing factor in both minor and serious health problems; it can affect nutrition, dental and psychological health. Some common problems associated with xerostomia include a constant sore throat, burning sensation, difficulties in speaking and swallowing, hoarseness and dry nasal passages. If left untreated, xerostomia causes a decrease in oral pH, and significantly increases the development of plaque and dental caries. Oral candidosis is one of the most common infections seen in association with xerostomia.

In view of the above, lubricating compositions for oral use which provide beneficial mouth feel and possess good substantivity would be advantageous.

WO 96/19190 relates to oral compositions comprising a dimethicone copolyol, and is described as being an antiplaque and anti-stain agent.

WO 96/33693 relates to oral compositions comprising combinations of a dimethicone copolyol and a silicone polyol surfactant. The silicone polyol is added in order to facilitate the dispersion of the dimethicone copolyol.

Surprisingly, it has now been found that buccal cavity lubrication may be achieved by the use of an oral care composition comprising water soluble silicone polyols which have good substantivity.

Accordingly, the present invention provides an oral care composition for lubrication of the buccal cavity, comprising a water soluble silicone polyol polymer of formula (I) and an orally acceptable carrier or excipient,

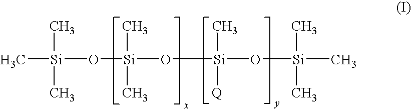

wherein Q is a C₁ to C₅ alkylene group mono or di-substituted by O(C₂H₄O)ₘ—(C₃H₆O)ₙR,
Q has a molecular weight of 200 to 20,000 kDa,
the ratio of m to n is from 10:1 to 1:10,
R is H, or C₁-C₃ alkyl,
x is 1-150 and
y is 1-80.

Q is preferably a propylene group mono or di-substituted by O(C₂H₄O)ₘ—(C₃H₆O)ₙR as hereinbefore described.

The alkyl groups may be branched or linear.

Suitably the ratio of m to n is from 5:1 to 1:5, and more suitably from 3.5:1 to 1:3.5.

Suitably the molecular weight of Q is from 700 to 12,000 kDa for example from 1200 to 10,000 kDa.

The compositions of the present invention show improved lubrication and substantivity in the buccal cavity in comparison to either traditional hydrophilic polymers or dimethicone, which is not soluble in water.

Examples of some suitable water soluble silicone polyol polymers include ABIL B 8852 (PEG/PPG-4/12 Dimethicone), ABIL B 8832 (Bis-PEG/PPG-20/20 Dimethicone), ABIL B 88184 (PEG/PPG-20/6 Dimethicone), ABIL 8851 (PEG/PPG-14/4 Dimethicone), ABIL 8863 (PEG/PPG-20/20 Dimethicone), GP-215 and GP-209. Preferred water soluble silicone polyol polymers are ABIL B 8852 (PEG/PPG-4/12 Dimethicone), ABIL B 8832 (Bis-PEG/PPG-20/20 Dimethicone), ABIL B 88184 (PEG/PPG-20/6 Dimethicone) or GP-215. (ABIL Silicones: Goldschmidt AG, Goldschmidtstr. 100, 45127, Essen, GP Silicones: Genesee Polymers Corporation, G-4099 South Dort Highway, Burton, Mich. 48529).

Suitably the aqueous solubility of a water soluble polyol polymer at 25° C. is at least 2 mg/ml, more suitably 10 mg/ml, preferably 20 mg/ml. The advantage of being water soluble is that the silicone polyol polymers provide good lubricity and substantivity compared to dimethicone which is not soluble in water, and which is therefore readily removed from the mouth in use.

The oral compositions of the present invention are typically formulated in the form of toothpastes, sprays, mouthwashes, gels, lozenges, chewing gums, tablets, pastilles, instant powders, oral strips and buccal patches. Preferred compositions of the present invention are mouth sprays, mouthwashes, lozenges and oral gels.

Compositions of the present invention comprise from 0.05-50% w/w of water soluble silicone polyol polymer, preferably 0.1-20% w/w, more preferably 0.5-10% w/w.

Suitable orally acceptable carriers and excipients include abrasive polishing materials (especially for a dentifrice), flavouring agents, humectants, binders, sweetening agents, surfactants, preservatives, buffering agents, colouring agents and water.

Suitable humectants for use in compositions of the invention include glycerine, sorbitol, xylitol, propylene glycol or polyethylene glycol, or mixtures thereof; which humectant may be present in the range from 5 to 70% w/w.

Suitable flavouring agents for use in the present invention include peppermint, spearmint, and fruit flavours. Flavouring agents provide an additional benefit in stimulating salivary flow, which helps alleviate the symptoms of dry mouth. If desired, additional salivary stimulants can be included such as edible organic acids, e.g. citric acid.

Suitable preservatives for use in the invention include parabens (methyl and propyl parabens), sodium benzoate, and potassium sorbate.

Suitable buffering agents for use in the invention include phosphate buffers such as disodium phosphate, sodium phosphate or citrate buffers.

Suitable surfactants for use in the invention include polyethyleneglycols (PEG), hydrogenated caster oils, sorbitan esters, polyethylene-polypropylene tri-block copolymers (such as Poloxamers™).

Compositions of the present invention may contain additional ingredients suitable including remineralising agents, antimicrobial agents, anti-caries agents, anticalculus agents, moisturising agents, breath freshening agents and desensitising agents.

Suitable antimicrobial agents for use in the invention include chlorhexidine, cetylpyridinium chloride, zinc salts and triclosan. Preferred antimicrobial agents are cetylpyridinium chloride, chlorhexidine and zinc salts.

Suitable anti-caries agents for use in the invention include a source of fluoride ions such as an alkali metal salt, for example sodium fluoride, or sodium monofluorophosphate, tin (II) fluoride or an amine fluoride salt such as Olaflur or Decaflur. Suitably the composition will comprise between 1 and 2500 ppm of fluoride ions.

Compositions according to the present invention may be prepared by admixing the ingredients in the appropriate relative amounts in any order that is convenient and if necessary including a buffering agent to adjust the pH to give the final desired value.

The compositions according to the present invention will have a pH which is orally acceptable, typically ranging from about pH 5 to 10 and more preferable pH 5.5 to 8.

Mouthwash and mouth spray compositions may be provided in a “ready to use” form; as a concentrated solution for dilution by the user immediately prior to use; or in solid form, such as a tablet or as instant powder in a sachet, for dissolution by the user immediately prior to use. Instand powders and tablets may be formulated to provide, on dissolution, a still mouthwash, or, by the incorporation of a suitable effervescent couple, for instance sodium carbonate/bicarbonate and citric acid, an effervescent mouthwash.

The compositions of the present invention are of use in lubricating the buccal cavity, alleviating the symptoms of xerostomia, and providing improved mouth feel. Effective lubrication of the buccal cavity advantageously aids in the hydration of the buccal cavity. Accordingly the compositions of the present invention are of use in lubricating and hydrating the buccal cavity.

The present invention also provides a method for lubricating and hydrating the buccal cavity and/or alleviating the symptoms of xerostomia in a subject in need thereof, wherein said method comprises administration of a therapeutically effective amount of a composition as hereinbefore described.

The invention is illustrated by way of the following examples:

### EXAMPLE 1

Thermogavimetric Analysis (TGA) is a well known technique for assessing the weight percentage of organic material in a sample. The use of TGA as a model for substantivity relies upon the fact that organic compounds are combustible at much lower temperatures than inorganic materials. The surface chosen for this model was high resolution hydroxyapatite powder, considered to be a reasonable substrate to mimic interactions of polymers with oral surfaces.

A suspension of high resolution hydroxyapatite powder was created from an aqueous solution of test polymer. This suspension was stirred overnight in order to evaporate the water. A sample of the solid obtained from this first step was analyzed by TGA to obtain a baseline measurement. From the remaining polymer/HA solid mixture, three equal portions were separately stirred in CaCl₂ solutions for varying lengths of time. After stirring, centrifugation was performed to recover the solid, and TGA was performed on this material. The percentage of each polymer remaining was calculated at each time point. The chart below shows examples of the results obtained for a selection of silicone polymers using this model. All the silicones shown are water soluble except dimethicone which is included to act as a benchmark. The PEG/PPG Dimethicone ABIL polymers in particular show improved substantivity compared to dimethicone, despite the fact that dimethicone is insoluble in water. The in vitro substantivity model favours insoluble polymers to a degree, as their removal from the hydroxyapatite surface is hindered by their insolubility. Despite this fact, a number of the soluble silicones show enhanced substantivity compared to dimethicone. Furthermore the water soluble silicones are significantly easier to formulate than insoluble silicones.

### EXAMPLE 2

An exploratory in vivo study was run with one of these polymers (Abil 88184). It was a single centre, double blind, randomised crossover study in 30 subjects who self reported a feeling of mouth dryness, most likely due to medications. An aqueous solution of the test polymer at two different concentrations (0.25% and 0.75%) was compared with water alone and consisted of three days home use. The test solutions were used at three specified times throughout the day and a Duration of Benefit Questionnaire and Daily Diary card was completed. Each three day home use period was separated by a minimum 1 day maintenance phase.

The duration of benefit of mouth moisturisation was recorded on an eight point scale

| Duration of Moisturisation | Score |
|---|---|
| Not at all | 1 |
| Up to 30 minutes | 2 |
| More than 30 minutes up to 1 hour | 3 |
| More than 1 hour up to 2 hours | 4 |
| More than 2 hour up to 3 hours | 5 |
| More than 3 hour up to 4 hours | 6 |
| More than 4 hour up to 12 hours | 7 |
| More than 12 hour up to 24 hours | 8 |

After 3 days usage both levels of Abil 88184 gave significantly increased duration of Mouth moisturisation when compared to water on the above scale:

| Comparison | Difference | p-Value |
|---|---|---|
| 0.25% Abil 88184 vs water | 1.21 | <0.0001 |
| 0.75% Abil 88184 vs water | 1.26 | <0.0001 |
| 0.25% Abil 88184 vs 0.75% Abil 88184 | 0.05 | 0.8527 |

1. An oral care composition for lubrication of the buccal cavity, comprising a water soluble silicone polyol polymer of formula (I) and an orally acceptable carrier or excipient, 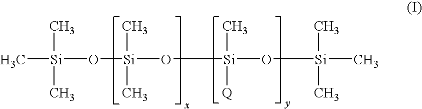
wherein Q is a C₁ to C₅ alkylene group mono or di-substituted by O(C₂H₄O)ₘ—(C₃H₆O)ₙR,
Q has a molecular weight of 200 to 20,000 kDa,
the ratio of m to n is from 10:1 to 1:10,
R is H, or C₁-C₃ alkyl,
x is 1-150 and
y is 1-80.

2. An oral care composition as claimed in claim 1 wherein Q is a propylene group mono or di-substituted by O(C₂H₄O)ₘ—(C₃H₆O)ₙR.

3. An oral care composition as claimed in claim 1 wherein the ratio of m to n is 5:1 to 1:5.

4. An oral care composition as claimed in claim 1 wherein the ratio of m to n is 3.5:1 to 1:3.5.

5. An oral care composition as claimed in claim 1 wherein the molecular weight of Q is from 700 to 12000 kDa.

6. An oral care composition as claimed in claim 1 wherein the molecular weight of Q is from 1200 to 10,000 kDa.

7. An oral care composition as claimed in claim 1 wherein the water soluble silicone polyol is selected from ABIL B 8852, ABIL B 8832, ABIL B 88184, ABIL 8851, ABIL 8863, GP-215 and GP-209.

8. An oral care composition as claimed in claim 7 wherein the water soluble silicone polyol is ABIL B 8852, ABIL B 8832, ABIL B 88184 or GP-215.

9. An oral care composition as claimed in claim 1 for hydration of the buccal cavity.

10-12. (canceled)

13. A method of lubricating the buccal cavity which comprises administering a therapeutically effective amount of an oral composition as defined in claim 1.

14. A method of lubricating and hydrating the buccal cavity which comprises administering a therapeutically effective amount of an oral composition as defined in claim 1.

15. A method of alleviating the symptoms of xerostomia which comprises administering a therapeutically effective amount of an oral composition as defined in claim 1.
